SEQUENCE LISTING (2 sequences)

SEQ ID NO: 1 - DNA, 21 bp, Artificial Sequence
  /note=“Description of Artificial Sequence Synthetic human telomeric G-guadruplex DNA sequence”
  misc_feature (1)..(1): 6-carboxyfluorescein (FAM) fluorescent label
  misc_feature (21)..(21): 6-carboxytetramethylrhodamine (TAMRA) fluorescent label
gggttagggttagggttaggg

SEQ ID NO: 2 - DNA, 21 bp, Artificial Sequence
  /note=“Description of Artificial Sequence Synthetic human telomeric duplex DNA sequence”
  misc_feature (1)..(1): 6-carboxyfluorescein (FAM) fluorescent label
  misc_feature (11)..(11): n is hexaethylene glycol
  misc_feature (21)..(21): 6-carboxytetramethylrhodamine (TAMRA) fluorescent label
tatagctatantatagctata

(19) United States
(12) Patent Application Publication
(10) Pub. No.: US 2019/0330197 A1
(43) Pub. Date: Oct. 31, 2019

(54) SUBSTITUTED NAPHTHALENE DIIMIDES AND THEIR USE

(71) Applicant: UCL Business PLC, London (GB)

(72) Inventors: Stephen Neidle, Bushey, Hertfordshire (GB); Chiara Marchetti, Hitchin, Hertfordshire (GB); Stephan Alexander Ohnmacht, Allschwil (CH)

(21) Appl. No.: 16/063,089

(22) PCT Filed: Dec. 14, 2016
(86) PCT No.: PCT/GB2016/053937

§ 371 Date: Jun. 15, 2018

(30) Foreign Application Priority Data
Dec. 15, 2015 (GB) 1522118.7

(51) Int. Cl. C07D 413/14; A61P 35/00

(52) U.S. Cl. CPC C07D 413/14; A61P 35/00

(57) ABSTRACT

A compound comprising the formula I that has DNA-quadruplex binding and stabilising activity and potential in treatment of pancreatic and other human cancers.

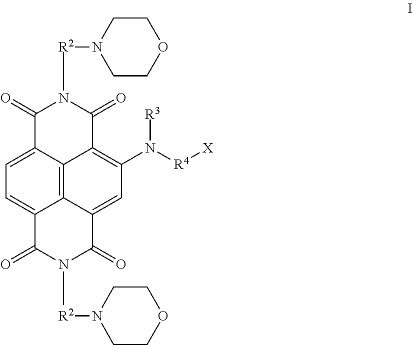

The present invention relates to naphthalene diimides, NDIs, and methods of synthesising them. The NDIs have DNA-quadruplex binding and stabilising activity and potential in treatment of pancreatic and other human cancers.

In WO2009/068916 we described tri- and tetra-substituted naphthalene diimides and processes for producing them. None of the exemplified products that were tri-substituted had different amino-functional ligands at equational and polar positions on the core ligand. The method said to be suited for producing tri-substituted compounds was based on the following schematic:—

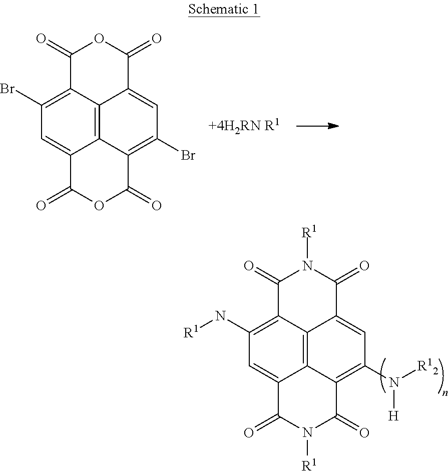

where R¹ is optionally substituted alkyl or aryl and n is 0 or 1. In practice a mixture of the tetra- (n=1) and tri-substituted (n=0) compounds was produced. All substituents, i.e. R¹ groups, are the same.

The specification describes methods for producing tetra-substituted compounds starting from the dichlorosubstituted analogue of the dibromo compound used above. The processes proceeded in one step, in which case the same H₂NR¹ reagent reacted at both anhydride groups and both—chlorine-substituted carbon to give 4 identical R¹ substituents on the product, or in two steps where in the first step a first reagent H₂NR² is reacted at both the anhydride groups and in a second step a second reagent H₂NR³ is reacted at both chlorine-substituted carbon atoms. Compounds with basic substituents on the imide substituent and/or on the aromatic rings have strong DNA quadruplex binding properties.

The tetra-substituted products, including products with groups R² different to groups R³, have been tested in WO2009/068916, US2014-0275065A and in Hampel S. M. et al., Bioorg. Med. Chem. Lett. (2010) 20, 6459-6463, Micco. M., et al., J. Med. Chem. (2013) 56, 2959-2974, Collie, G. W., et al., J.A.C.S. (2012) 134, 2723-2731, Gunaratnam, M. et al., J. Med. Chem. (2009) 52, 3774-3783, Gunaratnam, M. et al., Bioorg. Med. Chem. (2011) 19, 7151-7157 and Mitchell, T. et al., Biochemistry (2013) 52, 1429-1436 for their binding properties to quadruplexes of telomeres and also those found in the promoter region of some genes. The data show the effective down-regulation of several proteins, the promoters of whose genes are targeted by the diimides, and hence result in growth inhibition of several cell-lines from a panel of cancer cell-lines. We have proposed in these publications to investigate further the impact of changing the nature of the substituent groups and the basicity of the tertiary amine groups in the cationic-substituents, on binding specificity and strength, and to investigate the potential of the compounds in cancer treatment, by testing models of cancers including pancreatic cancer.

In Scientific Reports (2015) 5:11385, Ohnmacht, S. A., et al., disclose the activity of 4,9-bis((3-(4-methylpiperazine-1-yl)-propyl)amino)-2,7-bis(3-morpholinopropyl)benzo[Imn][3,8]phenanthroline-1,3,6,8(2H,7H)-tetraone, also known as MM41, in vivo in a mouse model of human pancreatic cancer.

Nadai, M., et al., in Int. J. Oncol. (2015) 46, 369-380, disclose a tri-substituted naphthalene diimide compound, having 2-dimethylamino ethyl groups substituted at each imido nitrogen atom and having, as the third substituent a 2-(4-hydroxy-3-dimethyl amino methyl phenyl)ethyl amino group substituted at the 4-position on the NDI core. It has activity stabilising the telomeric G-quadruplex, causing telomere dysfunction and telomerase down regulation. Global gene expression on a panel of cell lines showed modulation of genes implicated in telomere function and mechanisms of cancer. However the authors conclude that direct evidence for the biological relevance of G-4s in the cell context is still lacking.

The synthesis of the tri-substituted compound reported by Nadai et al. is disclosed in Doria et al, Org Biomol. Chem, (2012) 10, 2798-2806.

In the invention there is provided a new compound of formula I:

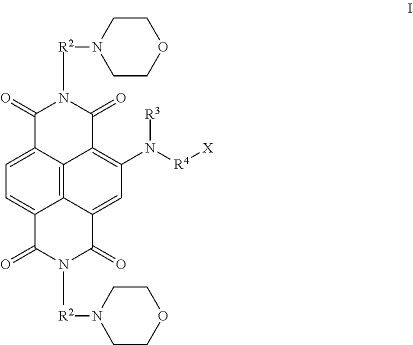

the groups R² are the same and are selected from the group consisting of straight and branched C₁₋₆-alkanediyl;

R³ is selected from the group consisting of H and C₁₋₆ alkyl;

R⁴ is selected from the group consisting of straight and branched C₁₋₆-alkanediyl and C₇₋₁₂ aralkane-diyl;

the group X is selected from the group consisting of halo, R¹, NR⁵₂, CONR⁶₂, COOR⁷, H and COR⁸, R¹ is selected from the group consisting of H, optionally substituted C₁₋₆ alkyl, optionally substituted C₅₋₇ cycloalkyl, C₅₋₇ heterocycloalkyl and aryl.

each R⁵ is selected from the group consisting of H, C₁₋₆ alkyl, aryl and C₇₋₁₂-arylkyl, or the groups R⁵ together with the N-atom to which they are attached form a N-containing, 5-7 membered heterocyclic group;

the groups R⁶ are each selected from H and C₁₋₆ alkyl groups or the groups R⁶ together with the N atom to which they are attached form a 5-7 membered heterocyclic ring;

R⁷ is selected from the group consisting of optionally substituted C₁₋₆ alkyl, C₇₋₁₂ aralkyl, and aryl;

R⁸ is selected from the group consisting of optionally substituted C₁₋₆-alkyl, C₇₋₁₂-aralkyl and aryl;

provided that when X is

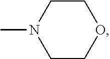

R⁴ is different to R²;

and salts, hydrates and solvates thereof.

Preferred definitions for the variable groups appear from the claims depending from claim 1.

Compounds where X is an amine group, i.e. a group NR⁵₂ are particularly preferred. Of such compounds, those where the two groups R⁵ are linked to form a heterocycle are preferred as they seem to have useful cytotoxic activity in cancer cell line tests.

We have created a range of acid addition salts of the preferred compound, where X is 2-(pyrrolidin-y-yl)ethyl, to provide control of the aqueous solubility of the compounds. Preferably the salt is a carboxylate, e.g. a di-carboxylate, a tricarboxylate, such as formate or acetate. It may be a mixed salt, e.g. of HCl and a carboxylate.

The invention further provides the new compounds for use in a method of treatment of an animal to inhibit the growth of a solid tumour, or to reduce the size of a solid tumour, for instance pancreatic tumour.

The invention also provides compositions containing the new compound and a diluent or carrier. The compositions are preferably pharmaceutical compositions and the carrier is then pharmaceutically acceptable.

The invention also provides the above-mentioned compositions further comprising a compound of formula II:

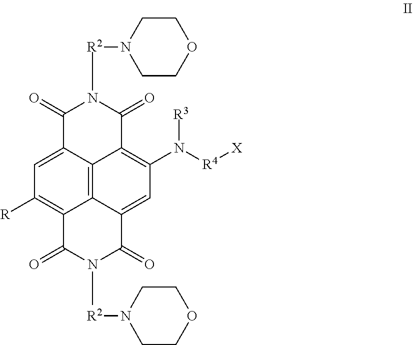

wherein R is NH₂ or N(R¹³)R¹⁴X¹, wherein

R¹³ is selected from the same groups as R³,

R¹⁴ is selected from the same groups as R⁴,

X¹ is selected from the same groups as X,

provided that the compound of formula II is present in an amount no higher than 50% by weight of the compound of formula I.

In a second aspect of the invention there is provided a method for synthesis of a substituted naphthalene diimide compound comprising the step of reacting a compound of formula III:

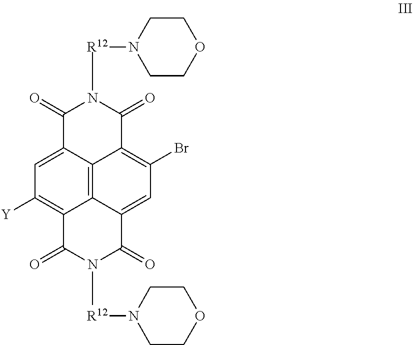

wherein Br is bromo;

Y is H or Br;

the group R¹² are the same and are selected from the group consisting of straight and branched C₁₋₆ alkanediyl;

in a nucleophilic substitution reaction with an amine of formula IV:

R¹³HNR¹⁴X² IV

wherein R¹³ is selected from the group consisting of H and C₁₋₆ alkyl;

R¹⁴ is selected from the group consisting of straight and branched C₁₋₆ alkanediyl and C₇₋₁₂ aralkanediyl;

X² is selected from the group consisting of halogens, OR, NR¹⁵₂, CONR¹⁶₂, COOR¹⁷, SH and COR¹⁸;

R¹¹ is selected from is selected from the group consisting of H, optionally substituted C₁₋₆ alkyl, optionally substituted C₅₋₇ cycloalkyl, C₅₋₇ heterocycloalkyl and aryl;

each R¹⁵ is selected from the group consisting of H, C₁₋₆ alkyl, aryl and C₇₋₁₂ aralkyl, or the groups R¹⁵ together with the N-atom to which they are attached form a saturated heterocyclic ring of 5-7 atoms;

each R¹⁶ is selected from the group consisting of H and C₁₋₆ alkyl groups or the groups R¹⁶ together with the N atom to which they are attached form a 5-7 membered heterocyclic ring;

R¹⁷ is selected from the group consisting of optionally substituted C₁₋₆ alkyl, C₇₋₁₂ aralkyl and aryl;

R¹⁸ is selected from the group consisting of optionally substituted C₁₋₆ alkyl, C₇₋₁₂ aralkyl and aryl;

whereby the Br atom, one of the Br atoms or each Br atom is substituted by the nucleophilic amine nitrogen of the amine reagent to form the substituted NDI compound.

Preferred aspects of the method appear from claims depending from claim 12.

Thus the method of the invention comprises the step of reacting a brominated naphthalene diimide with an amine reagent of formula IV in an aromatic nucleophilic substitution reaction whereby the bromine atom is replaced by an amino group N(R¹³)R¹⁴X². The starting diimide may be a mono-bromo compound or (Y is H) a dibromo compound (Y is Br) or a mixture of both. Where the starting diimide comprises a dibromo compound, the aromatic nucleophilic substitution reaction may result in both bromine atoms being replaced by an amine group or just one of them. The second bromine atom may be replaced by H, in which event the product is a tri-substituted compound according to the first aspect of the invention. This occurs more readily at high temperatures. It is preferable to separate a mixture of both, for example by using column chromatography. Where the diimide starting material has one bromine substituent the product will be the new compound of the first aspect of the invention. The product may comprise a mixture of substituted NDIs and may also contain unreacted NDI. Where the product comprises a mixture of substituted NDIs, with or without unreacted NDIs, no higher than 50% by weight of the tetra-substituted NDIs compared to the tri-substituted NDIs may be present.

The process step of reacting a compound of formula III wherein Y is H is particularly preferred. If it is desired to form the novel tri-substituted compounds from starting compound of formula III wherein Y is Br, it may be desirable to use relatively high temperature conditions since this may optimise replacement of the second Br atom by H. Generally, however such high temperature conditions could lead to unwanted side reactions involving ring opening of the imide rings. Preferably therefore the process is carried out under reflex.

The process usually involves a purification step for isolating the desired end product from the product mixture. It is an advantage of the process of the present invention that column chromatography can be used as a facile purification step. This is rendered possible by selection of reaction conditions and reagents for this step and for the preliminary steps of synthesising the imido and its precursor brominated tetracarboxylic anhydride and the precursor anhydride for that.

The process of the invention preferably involves the preliminary steps as defined in the accompanying claims 20 and 23 and claims dependent thereon and as exemplified in the worked examples.

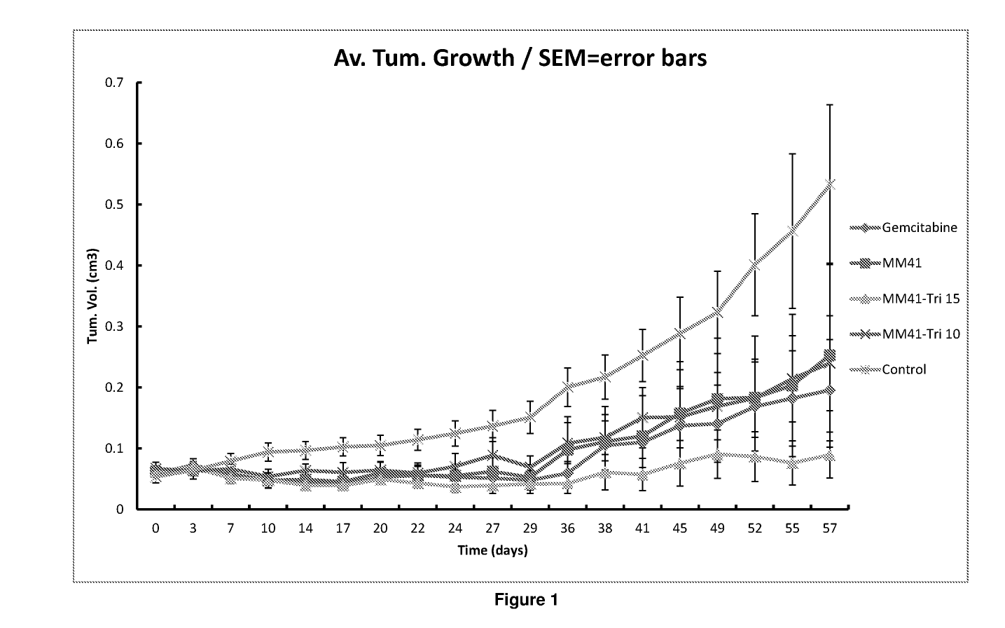

The novel compounds of the present invention, being tri-substituted, have lower molecular weight than the compounds disclosed in our earlier publication US2014-0275062, which are tetra-substituted. When compared with the tetra-substituted compounds, at the same molar concentration, in a DNA quadruplex stabilisation test, using FRET analysis, the compounds are found to produce diminished stabilisation of a series of DNA quadruplexes of potential relevance to tumour growth. Furthermore, when tested for cytotoxicity against a panel of human cancer cell-lines, the IC₅₀ values are comparable to those of our earlier compound MM41, a tetra-substituted compound. We have surprisingly found that when tested in an in vivo test, a xenograft mouse model for pancreatic cancer, using MIA-PaCa2 pancreatic tumour cell line, the results were better than a regimen using the same concentration of the tetra-substituted compound, indeed better than the current therapeutic standard of care in pancreatic cancer, gemcitabine (FIG. 1). Animals did not have any significant weight loss on treatment. When tested at a lower concentration, the results on tumour growth are equivalent to the test concentration for the MM41 compound used in our previous trials. These data lead to an expectation that the compounds will be therapeutically effective in treatment of solid tumours, for instance pancreatic tumours.

Surprisingly it has been found that MIA-PaCa2 pancreatic cancer cells when treated with the novel compounds of the present invention, show changes in gene expression in a comparatively small number of genes, the majority of which have putative quadruplex-forming sequences in their promoters, consistent with the concept that the novel compounds selectively target these nuclear genes.

Also surprisingly, a number of these targeted genes are associated with increased patient survival where their expression in humans is down-regulated, supporting the concept that the novel compounds of the present invention may have utility in the treatment of human cancers.

The compounds of the present invention may be provided in the form of pharmaceutically acceptable compositions. The compositions may be administered by any route. For chemotherapy of tumours, the compositions are most conveniently administered intravenously. The compounds of the present invention, especially when presented in the form of acid addition salts, for instance where some or all of the basic amine groups are converted to salt form, are water soluble and have approximately neutral pH. As such these salts are suitable for administration in the form of aqueous solution, which would be appropriate for intravenous administration. The pharmaceutical aqueous solutions preferably comprise 1 to 500 mg/l, of the compound.

The compounds of the present invention may be provided in a form suitable to be made up into pharmaceutical compositions, for instance, in dried, rehydratable form, for instance with carrier or diluent. Such dried forms may be produced by crystallisation and/or evaporation. Alternatively, the compounds may be presented as concentrates, for instance in water or an organic, pharmaceutically acceptable, solvent for dilution before administration.

When used as treatment for existing tumours, the compounds of the present invention may be administered using regimens developed for chemotherapeutic agents.

The invention is further illustrated in the accompanying examples.

### EXAMPLES

A series of trisubstituted naphthalene diimides have been synthesised and evaluated as G-quadruplex ligands, and as potential anti-cancer agents. A new synthetic procedure has been developed, as detailed below. Compound 4c in Schematic 2 has been identified as a lead compound at present:

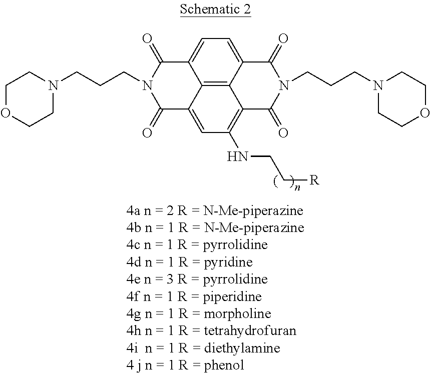

### Chemistry

All chemicals, reagents, and solvents were purchased from Sigma-Aldrich, Alfa Aesar, Lancaster Synthesis, and Fluorochem (UK) and used without further purification. Solvents were supplied by VWR and Fisher Scientific. Column chromatography was performed using BDH silica gel (BDH 153325P). Chromatography was conducted under medium pressure in glass columns or using a Biotage SP4 instrument in prepacked columns (FLASH Silica columns (40-63 μm, 60 Å)). HPLC analysis was carried out with a Gilson apparatus combining a 322 pump and an Agilent 1100 series detector, using a C18 5 μm (100 mm×4.6 mm) column (41622271 (W), YMC, Japan), at a flow of 1 mL/min. Preparative HPLC was carried out with a Gilson apparatus combining a 322 pump and a UV/vis-155 detector with detection at 280 nm, using a C18 5 μm (100 mm×20 mm) column (201022272) (W), YMC, Japan, at a flow of 20 mL/min. Water and methanol with 0.1% formic acid were used as solvents for HPLC. For the purification of compounds 4a-4i, the following method was used: 100% aqueous solution for 5 min after injection, then gradually decreased to 60% aqueous solution over 25 min.

For compounds 3a and 3b, the following method was used: 100% aqueous solution for 2 min after injection, gradually decreased to 20% solution over 17 min. For the HPLC purity analysis of compounds 4a-4i, the method used was: 100% aqueous solution for 5 min after injection, to 60% w/v aqueous solution over 18 min as well as 100% aqueous for 5 min after injection, to 60% w/v aqueous over 43 min. Purity for final compounds was greater than 95% (HPLC, 280 nm). NMR spectra were recorded at 400 MHz or 500 MHz on a Bruker spectrometer in CDCl₃ (with 0.05% TMS, Cambridge Isotope Laboratories, USA). NMR spectra were analyzed with MestReC 4.5.6.0 with chemical shifts using TMS as a standard (δ=0 ppm). NMR multiplicity abbreviations are s (singlet), bs (broad singlet), d (doublet), t (triplet), q (quartet), 5q (quintet), and m (multiplet). Coupling constants J are reported as observed in hertz (Hz). High resolution mass spectra (HRMS) were measured on a Micromass Q-TTOF Ultima global tandem mass spectrometer run under electrospray ionization (ESI), and processed using the MassLab 3.2 software. For compounds 2a and 2b, no ¹H and ¹³C NMRs were obtained due to solubility issues.

### Reference Method 1

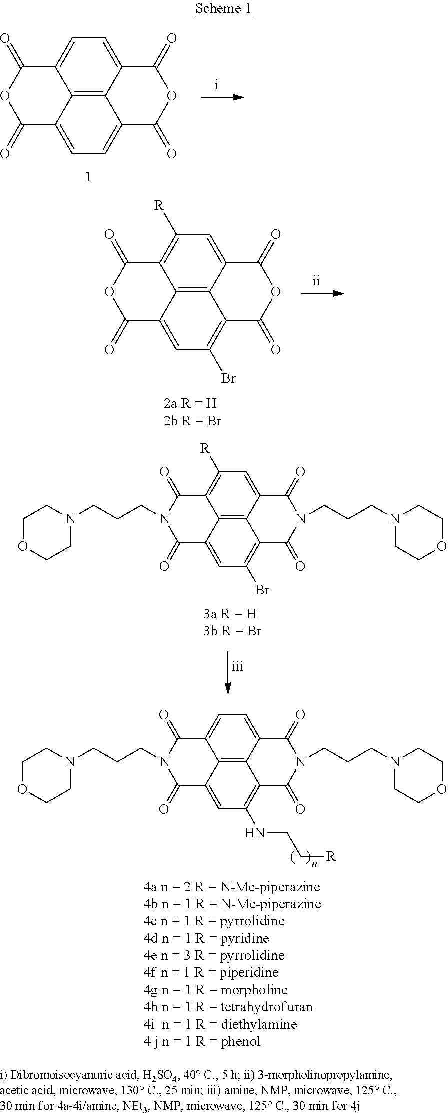

Compounds 4a-4j were initially synthesized using the procedure in Scheme 1.

### 2-Bromo-1,4,5,8-naphthalenetetracarboxylic acid dianhydride (2a) and 2,6-Dibromo-1,4,5,8-naphthalenetetracarboxylic acid dianhydride (2b)

Naphthalene dianhydride (1) (1 g, 3.72 mmol) was dissolved in concentrated sulphuric acid (96%) (38 ml). A solution of dibromoisocyanuric acid (1.07 g, 3.72 mmol) in concentrated sulphuric acid (18.5 ml) was added dropwise into it over a 4 h period. The mixture was stirred for a further 1 h and then poured onto ice (500 ml). The yellow solid that formed was filtered, washed with 0.5 M HCl in water (2×10 ml) and dried under vacuum. The yellow solid obtained, containing a mixture of 1, 2a and 2b, was used without further purification. We did not separate 2a and 2b because of the low solubility of those compounds. Also no NMR data were obtained due to solubility issues.

### N,N′-Bis(3-(morpholino)propylamino)-2-bromo-1,4,5,8-naphthalenetetracarboxylic acid diimide (3a) and N,N′-Bis(3-(morpholino)propylamino)-2,6-dibromo-1,4,5,8-naphthalenetetracarboxylic acid diimide (3b)

Compound 2 (mixture of 2a and 2b) (250 mg, 0.720 mmol) was suspended with sonication in glacial acetic acid (3 ml) in a microwave reaction vessel. 3-morpholinopropylamine (311 mg, 316 μL, 2.16 mmol) was added dropwise to the stirring mixture. The reaction tube was sealed and treated for 25 min at 130° C. in the microwave. After solvent removal, the residue was purified through preparative HPLC, to give the derivatives 3a and 3b as brown solids.

Yield 3a (48 mg, 0.08 mmol, 11.1%): ¹H NMR (400 MHz, CDCl₃) δ 2.08-2.13 (m, 4H), 2.77-2.83 (m, 12H), 3.72-3.76 (m, 8H), 4.25-4.31 (q, 4H), 8.75 (d, 1H, J=8 Hz), 8.80 (d, 1H, J=7.6 Hz), 8.92 (s, 1H). ¹³C NMR (100 MHz, CDCl3, TMS) δ 23.31, 23.44, 38.81, 39.11, 52.50, 55.51, 55.45, 65.37, 65.41, 123.97, 125.75, 125.95, 126.01, 126.84, 128.68, 128.76, 130.73, 131.71, 138.44, 161.17, 161.87, 161.98, 162.56. HRMS (ES⁺) calculated C₂₈H₃₁BrN₄O₆[M+H]⁺ 600.1543. found: 600.1536.

Yield 3b (49 mg, 0.0722 mmol, 10.0%): ¹H NMR (400 MHz, CDCl₃) δ: 2.00-1.92 (m, 4H), 2.45-2.37 (m, 8H), 2.53-2.50 (m, 4H), 3.54-3.52 (m, 8H), 4.33-4.28 (m, 4H), 8.99-8.76 (s, 2H). ¹³C NMR (100 MHz, CDCl3, TMS) δ 21.7, 38.5, 51.8, 54.7, 63.7, 123.4, 124.5, 126.7, 128.2, 138.5, 161.4, 161.5. HRMS (ES+) calculated C₂₈H₃₀Br₂N₄O₆ [M+H]⁺ 679.0603. found: 679.0593.

### 4-((3-(4-methylpiperazin-1-yl)propyl)amino)-2,7-bis(3-morpholinopropyl)benzo[Imn][3,8]phenanthroline-1,3,6,8(2H,7H)-tetraone (4a)

A mixture of compounds 3a and 3b (150 mg, 0.25 mmol), 1-(3-aminopropyl)-4-methylpiperazine (78 mg, 85 μl, 0.50 mmol) and NMP (2 ml) were suspended in a microwave vessel. The tube was sealed with a rubber cup and heated to 125° C. for 30 minutes under microwave irradiation. After solvent removal, the crude mixture was purified by preparative HPLC to give the formate salt 4a as a red solid. Yield 4a (52 mg, 0.072 mmol, 28.8%). ¹H NMR (400 MHz, CDCl₃) δ 1.99-2.09 (m, 6H), 2.62-2.80 (m, 17H), 3.01 (m, 8H), 3.68-3.76 (m, 10H), 4.20-4.26 (m, 4H), 8.23 (s, 1H), 8.31 (d, 1H, J=7.6 Hz), 8.61 (d, 1H, J=7.6 Hz), 10.16 (t, 1H, exch D₂O, J=5.8 Hz). ¹³C NMR (100 MHz, CDCl₃) δ 23.7, 23.9, 38.2, 38.8, 41.1, 43.3, 50.2, 52.5, 52.7, 53.1, 54.6, 55.7, 65.5, 65.7, 99.9, 119.4, 119.8, 123.4, 124.6, 126.1, 127.9, 129.5, 131.4, 152.3, 163.0, 163.3, 166.0, 166.4. HRMS (ES⁺) calculated for (M+H)⁺ C₃₆H₄₉N₇O₆ 676.3823, found 676.3825.

### 4-((2-(4-methylpiperazin-1-yl)ethyl)amino)-2,7-bis(3-morpholinopropyl)benzo[Imn][3,8]phenanthroline-1,3,6,8(2H,7H)-tetraone (4b)

A mixture of compounds 3a and 3b (150 mg, 0.25 mmol), 1-(2-aminoethyl)-4-methylpiperazine (72 mg, 75 μl, 0.50 mmol) and NMP (2 ml) were suspended in a microwave vessel. The tube was sealed with a rubber cup and heated to 125° C. for 30 minutes under microwave irradiation. After solvent removal, the crude mixture was purified by preparative HPLC to give the formate salt 4b as a red solid. Yield 4b (9.4 mg, 0.013 mmol, 5.3%). ¹H NMR (400 MHz, CDCl₃) δ 1.96-2.02 (m, 4H), 2.53-2.68 (m, 21H), 3.03-3.05 (m, 4H), 3.62 (t, 4H, J=4.6 Hz), 3.67-3.72 (m, 6H), 4.23-4.28 (m, 4H), 8.21 (s, 1H), 8.34 (d, 1H, J=7.6 Hz), 8.64 (d, 1H, J=8 Hz), 10.28 (t, 1H, exch D₂O, J=4.8 Hz); ¹³C NMR (100 MHz, CDCl₃) 24.0, 38.3, 39.1, 40.2, 43.7, 50.4, 52.9, 53.1, 53.6, 55.8, 56.0, 56.0, 66.0, 66.3, 100.3, 119.5, 120.0, 123.6, 124.6, 126.3, 128.0, 129.5, 131.4, 152.1, 163.0, 163.1, 163.3, 165.9. HRMS (ES⁺) calculated for (M+H)⁺ C₃₅H₄₇N₇O₆ 662.3680, found 662.3666.

### 2,7-bis(3-morpholinopropyl)-4-((2-(pyrrolidin-1-yl)ethyl)amino)benzo[Imn][3,8]phenanthroline-1,3,6,8(2H,7H)-tetraone (4c)

A mixture of compounds 3a and 3b (150 mg, 0.25 mmol), 1-(2-aminoethyl)-pyrrolidine (57 mg, 63 μl, 0.50 mmol) and NMP (2 ml) were suspended in a microwave vessel. The tube was sealed with a rubber cup and heated to 125° C. for 30 minutes under microwave irradiation. After solvent removal, the crude mixture was purified by preparative HPLC to give the formate salt 4c as a red solid. Yield 4c (25 mg, 0.037 mmol, 14.8%). ¹H NMR (400 MHz, CDCl₃) δ ¹H NMR (400 MHz, CDCl₃) δ 2.02-2.04 (m, 8H), 2.68-2.78 (m, 12H), 3.19 (t, 4H, J=6.4 Hz), 3.32 (t, 2H, J=6.6 Hz), 3.71 (t, 4H, J=4.8 Hz), 3.76 (t, 4H, J=4.6 Hz), 4.01-4.02 (m, 2H), 4.22 (t, 4H, J=7 Hz), 8.24 (s, 1H), 8.28 (d, 1H, J=7.6 Hz), 8.56 (d, 1H, J=8 Hz), 10.17 (t, 1H, exch D₂O, J=4.2 Hz). ¹³C NMR (100 MHz, CDCl₃) δ 23.3, 23.6, 23.7, 38.2, 38.9, 40.1, 52.6, 52.7, 53.9, 53.9, 55.8, 65.6, 65.8, 100.7, 119.1, 119.5, 123.5, 124.9, 126.1, 128.0, 129.2, 131.4, 151.8, 162.8, 163.2, 165.9, 166.5. HRMS (ES⁺) calculated for (M+H)⁺ C₃₄H₄₄N₆O₆ 633.3389, found 633.3401.

### 2,7-bis(3-morpholinopropyl)-4-((2-(pyridin-2-yl)ethyl)amino)benzo[Imn][3,8]phenanthroline-1,3,6,8(2H,7H)-tetraone (4d)

A mixture of compounds 3a and 3b (150 mg, 0.25 mmol), 2-(2-aminoethyl)pyridine (61 mg, 59 μl, 0.50 mmol) and NMP (2 ml) were suspended in a microwave vessel. The tube was sealed with a rubber cup and heated to 125° C. for 30 minutes under microwave irradiation. After solvent removal, the crude mixture was purified by preparative HPLC to give the formate salt 4d as a red solid. Yield 4d (12 mg, 0.017 mmol, 7%). ¹H NMR (400 MHz, CDCl₃) δ 1.90-1.96 (m, 4H), 2.49-2.58 (m, 12H), 3.22 (t, 2H, J=7 Hz), 3.57 (t, 4H, J=4.6 Hz), 3.62 (t, 4H, J=4.6 Hz), 3.97-4.02 (m, 2H), 4.16-4.20 (m, 4H), 7.11-7.15 (m, 1H), 7.29-7.27 (m, 1H), 7.57-7.61 (d, 1H, J=7.8 Hz), 8.20 (s, 1H), 8.26 (d, 1H, J=8 Hz), 8.56 (d, 2H, J=8 Hz), 10.21 (t, 1H, exch D₂O, J=5.6 Hz); ¹³C NMR (100 MHz, CDCl₃) δ 24.2, 37.7, 38.6, 39.2, 42.8, 53.1, 53.3, 56.2, 66.5, 66.5, 100.1, 119.8, 121.9, 123.5, 123.6, 124.5, 126.2, 127.9, 129.5, 131.3, 136.8, 149.7, 151.9, 152.2, 157.8, 163.0, 163.1, 163.4, 166.1. HRMS (ES+) calculated for (M+H)+C₃₅H₄₀N₆O₆ 641.3090, found 641.3088.

### 2,7-bis(3-morpholinopropyl)-4-((4-(pyrrolidin-1-yl)butyl)amino)benzo[Imn][3,8]phenanthroline-1,3,6,8(2H,7H)-tetraone (4e)

A mixture of compounds 3a and 3b (100 mg, 0.17 mmol), 4-(1-pyrrolidinyl)-1-butylamine (48 mg, 51 μl, 0.33 mmol) and NMP (1.5 ml) were suspended in a microwave vessel. The tube was sealed with a rubber cup and heated to 125° C. for 30 minutes under microwave irradiation. After solvent removal, the crude mixture was purified by preparative HPLC to give the formate salt 4e as a red solid. Yield 4e (17 mg, 0.024 mmol, 14%). ¹H NMR (400 MHz, CDCl₃) δ 1.94-2.11 (m, 12H), 2.59-2.77 (m, 12H), 3.14 (t, 2H, J=7.8 Hz), 3.28-3.29 (m, 4H), 3.63-3.70 (m, 6H), 3.76 (t, 4H, J=4.6 Hz), 4.23-4.27 (m, 4H), 8.17 (s, 1H), 8.34 (d, 1H, J=8 Hz), 8.64 (d, 1H, J=8 Hz), 10.10 (t, 1H, exch D₂O, J=5.4 Hz); ¹³C NMR (100 MHz, CDCl₃) δ 23.2, 23.4, 23.7, 23.8, 26.7, 38.3, 38.9, 42.4, 52.7, 52.8, 53.3, 54.7, 55.8, 55.9, 65.7, 65.9, 100.2, 119.5, 119.5, 123.6, 124.7, 126.3, 128.1, 129.5, 131.4, 152.3, 162.9, 163.1, 163.4, 166.2. HRMS (ES⁺) calculated for (M+H)⁺ C₃₆H₄₈N₆O₆ 661.3713, found 661.3713.

### 2,7-bis(3-morpholinopropyl)-4-((2-(piperidin-1-yl)ethyl)amino)benzo[Imn][3,8]phenanthroline-1,3,6,8(2H,7H)-tetraone (4f)

A mixture of compounds 3a and 3b (100 mg, 0.17 mmol), 1-(2-aminoethyl)piperidine (42 mg, 47 μl, 0.33 mmol) and NMP (1.5 ml) were suspended in a microwave vessel. The tube was sealed with a rubber cup and heated to 125° C. for 30 minutes under microwave irradiation. After solvent removal, the crude mixture was purified by preparative HPLC to give the formate salt 4f as a red solid. Yield 4f (13 mg, 0.018 mmol, 11%). ¹H NMR (400 MHz, CDCl₃) δ 1.57-1.60 (m, 2H), 1.69-1.75 (m, 4H), 1.95-2.00 (m, 4H), 2.51-2.68 (m, 16H), 2.87 (t, 2H, J=6.4 Hz), 3.61 (t, 4H, J=4.6 Hz), 3.67 (t, 4H, J=4.6 Hz), 3.76-3.80 (m, 2H), 4.22-4.28 (m, 4H), 8.18 (s, 1H), 8.31 (d, 1H, J=7.6 Hz), 8.61 (d, 1H, J=8 Hz), 10.24 (t, 1H, exch D₂O, J=5 Hz); ¹³C NMR (100 MHz, CDCl₃) δ23.8, 24.2, 25.2, 38.5, 39.2, 39.9, 53.1, 53.2, 54.3, 56.2, 56.8, 66.4, 66.5, 100.3, 199.5, 119.9, 123.6, 124.5, 126.2, 127.9, 129.5, 131.2, 152.0, 162.9, 163.1, 163.4, 165.9. HRMS (ES+) calculated for (M+H)+ C₃₅H₄₆N₆O₆ 647.3566, found 647.3557.

### 4-((2-morpholinoethyl)amino)-2,7-bis(3-morpholinopropyl)benzo[Imn][3,8]phenanthroline-1,3,6,8(2H,7H)-tetraone (4g)

A mixture of compounds 3a and 3b (60 mg, 0.1 mmol), 4-(2-aminoethyl)morpholine (26 mg, 30 μl, 0.2 mmol) and NMP (1 ml) were suspended in a microwave vessel. The tube was sealed with a rubber cup and heated to 125° C. for 30 minutes under microwave irradiation. After solvent removal, the crude mixture was purified by preparative HPLC to give the formate salt 4g as a red solid. Yield 4g (15 mg, 0.022 mmol, 22%). ¹H NMR (400 MHz, CDCl₃) δ 1.92-1.96 (m, 4H), 2.42-45 (m, 8H), 2.49-2.54 (m, 4H), 2.59 (t, 4H, J=4.4 Hz), 2.80 (t, 2H, J=6 Hz), 3.56 (m, 4H, J=4.4 Hz), 3.62 (t, 4H, J=4.4 Hz), 3.66-3.70 (m, 2H), 3.78 (t, 4H, J=4.4 Hz), 4.23-4.30 (m, 4H), 8.21 (s, 1H), 8.33 (d, 1H, J=7.6 Hz), 8.64 (d, 1H, J=8 Hz), 10.34 (t, 1H, exch D₂O, J=4.8 Hz); ¹³C NMR (100 MHz, CDCl₃) δ 24.4, 24.6, 38.7, 39.4, 40.1, 53.5, 53.6, 56.4, 56.5, 56.8, 66.93, 66.96, 66.99, 100.3, 119.5, 120.0, 123.7, 124.5, 126.2, 127.9, 129.5, 131.2, 152.1, 163.1, 163.5, 165.9. HRMS (ES⁺) calculated for (M+H)⁺ C₃₄H₄₄N₆O₇ 649.3376, found 649.3350.

### 4-((tetrahydrofuryl methyl)amino)-2,7-bis(3-morpholinopropyl) benzo[Imn][3,8]phenanthroline-1,3,6,8(2H,7H)-tetraone (4h)

A mixture of compounds 3a and 3b (60 mg, 0.1 mmol), tetrahydrofurfurylamine (21 mg, 21 μl, 0.2 mmol) and NMP (1 ml) were suspended in a microwave vessel. The tube was sealed with a rubber cup and heated to 125° C. for 30 minutes under microwave irradiation. After solvent removal, the crude mixture was purified by preparative HPLC to give the formate salt 4h as a red solid. Yield 4h (10 mg, 0.015 mmol, 15%). ¹H NMR (400 MHz, CDCl₃) δ 1.26-1.28 (m, 4H), 1.71-1.78 (m, 6H), 1.94-2.05 (m, 2H), 2.12-2.51 (m, 10H), 3.56-3.57 (m, 4H), 3.62-3.64 (m, 4H), 3.74-3.78 (m, 1H), 3.84-3.87 (m, 1H), 3.97-4.00 (m, 1H), 4.24-4.28 (m, 4H), 8.27 (s, 1H), 8.34 (d, 1H, J=7.6 Hz), 8.65 (d, 1H, J=7.6 Hz), 10.34 (t, 1H, exch D₂O, J=4.8 Hz); ¹³C NMR (100 MHz, CDCl₃) δ 24.4, 24.6, 25.9, 38.8, 39.4, 47.3, 53.6, 56.4, 56.5, 66.94, 66.97, 68.6, 100.2, 119.5, 120.0, 123.7, 124.5, 126.2, 127.9, 129.5, 131.2, 152.5, 163.1, 163.4, 166.2. HRMS (ES⁺) calculated for (M+H)⁺ C₃₃H₄₁N₅O₇ 620.3096, found 620.3084.

### 4-((2-(diethylamino)ethyl)amino)-2,7-bis(3-morpholinopropyl)benzo[Imn][3,8]phenanthroline-1,3,6,8(2H,7H)-tetraone (4i)

Compound 3a (24 mg, 0.04 mmol), N,N-diethylethylenediamine (9 mg, 11 μl, 0.08 mmol) and NMP (0.8 ml) were suspended in a microwave vessel. The tube was sealed with a rubber cup and heated to 125° C. for 30 minutes under microwave irradiation. After solvent removal, the crude mixture was purified through column chromatography using as eluent a mixture of CH₂Cl₂/MeOH/NH₃37% 9.5/0.5/0.03 to give compound 4i as a red solid. Yield 4i (15 mg, 0.022 mmol, 59%). ¹H NMR (400 MHz, CDCl₃) δ 1.11 (t, 6H, J=7 Hz), 1.46-1.47 (m, 4H), 1.90-2.53 (m, 12H), 2.64-2.69 (m, 4H), 2.86 (t, 2H, J=6.2 Hz), 3.56-3.57 (m, 4H), 3.61-3.65 (m, 6H), 4.23-4.30 (m, 4H), 8.21 (s, 1H), 8.31 (d, 1H, J=7.6 Hz), 8.62 (d, 1H, J=8 Hz), 10.27 (t, 1H, exch D₂O, J=5 Hz); ¹³C NMR (100 MHz, CDCl₃) δ 11.8, 24.4, 24.5, 38.6, 39.3, 47.1, 51.6, 53.5, 53.6, 56.5, 66.9, 100.1, 119.4, 120.2, 123.7, 124.3, 126.1, 127.8, 129.6, 131.2, 152.1, 163.12, 163.16, 163.5, 165.9. HRMS (ES⁺) calculated for (M+H)⁺ C₃₄H₄₆N₆O₆ 635.3552, found 635.3557.

### 4-((4-hydroxyphenethyl)amino)-2,7-bis(3-morpholinopropyl)benzo[Imn][3,8]phenanthroline-1,3,6,8(2H,7H)-tetraone (4j)

A mixture of compounds 3a and 3b (139 mg, 0.23 mmol), tyramine hydrochloride (80 mg, 0.46 mmol), triethylamine (47 mg, 65 μl, 0.46 mmol) and NMP (2 ml) were suspended in a microwave vessel. The tube was sealed with a rubber cup and heated to 125° C. for 30 minutes under microwave irradiation. After solvent removal, the crude mixture was purified through column chromatography using as eluent a mixture of CH₂Cl₂/MeOH 9.5/0.5 to give compound 4j as a red solid. Yield 4j (11 mg, 0.016 mmol, 7%). ¹H NMR (400 MHz, CDCl₃) δ 1.75-1.82 (m, 4H), 2.31-2.43 (m, 12H), 2.91 (t, 2H, J=6.6 Hz), 3.41-3.44 (m, 8H), 3.9-3.71 (m, 2H), 4.02 (m, 4H), 6.72 (d, 2H, J=8.4 Hz), 7.15 (d, 2H, J=8 Hz), 7.84 (s, 1H), 8.02 (d, 1H, J=8 Hz), 8.30 (d, 1H, J=8 Hz), 9.86 (brs, 1H, exch D₂O). ¹³C NMR (100 MHz, CDCl₃), δ 29.9, 30.9, 38.4, 38.9, 44.5, 53.4, 53.5, 56.2, 56.4, 66.5, 66.7, 94.4, 100.1, 116.1, 119.4, 120.2, 123.5, 124.5, 126.2, 129.0, 130.2, 131.3, 155.4, 162.9, 163.1, 163.5, 165.8. HRMS (ES⁺) calculated for (M+H)⁺ C₃₆H₄₁N₅O₇ 656.3074, found 656.3084.

### Reference Method 2

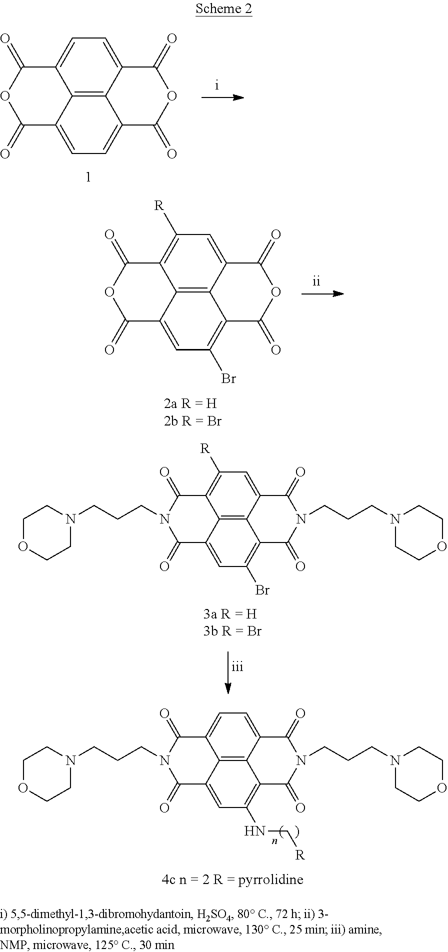

The synthesis of 4c was optimized according to Scheme 2. The process involves purification of the product mixture of step ii, to separate out 3a and 3b and no-Br side-products. This allowed easier purification of the products 4 by column chromatography. This also allowed scale up not possible with preparative HPLC as used in method 1. Different reaction conditions were used to obtain compound 3a. All the conditions used and the relative yields for compounds 3a and 3b are reported in Table 1.

TABLE 1
Reaction condition tested for the synthesis of intermediate 3a.

| 5,5-dimethyl-1,3- | | | | |
|---|---|---|---|---|
| dibromohydantoin | | Conditions I step | Yield % 3a | Yield % 3b |
| 0.55 | equiv | H₂SO₄, r.t., 12 h | 12 | 3 |
| 0.55 | equiv | H₂SO₄, r.t., 70 h | 16 | 5 |
| 0.55 | equiv | H₂SO₄, 80° C., 24 h | 21 | 2 |
| 0.55 | equiv | H₂SO₄, 80° C., 48 h | 26 | 5 |
| 0.55 | equiv | H₂SO₄, 80° C., 72 h | 35 | 6 |
| 1.0 | equiv | H₂SO₄, 80° C., 24 h | 28 | 13 |
| 1.0 | equiv | H₂SO₄, 80° C., 48 h | 22 | 33 |
| 1.5 | equiv | H₂SO₄, 50° C., 10 h | 26 | 25 |

### 2-Bromo-1,4,5,8-naphthalenetetracarboxylic acid dianhydride (2a) and 2,6-Dibromo-1,4,5,8-naphthalenetetracarboxylic acid dianhydride (2b)

Naphthalene dianhydride (1) (150 mg, 0.56 mmol) was slurried in sulphuric acid (1.5 ml) and the suspension obtained was stirred for 5 min at room temperature to allow the complete dissolution. 5,5-dimethyl-1,3-dibromohydantoin (88 mg, 0.308 mmol) was added slowly over a period of 1 h and the round bottom flask was tightly stopped to avoid the escape of bromine from the reaction mixture. The solution was stirred at 80° C. for 72 h and then poured onto ice (30 ml). The yellow solid formed was filtered, washed with water (2×10 ml) and dried under vacuum, yielding a mixture 2a and 2b. No NMR data were obtained due to solubility issues. The resulting mixture was used without further purification in the next step.

### N,N′-Bis(3-(morpholino)propylamino)-2-bromo-1,4,5,8-naphthalenetetracarboxylic acid diimide (3a)

Compounds 2a and 2b (194 mg, 0.56 mmol) were suspended with sonication in glacial acetic acid (1.5 ml) in a microwave reaction vessel. 3-morpholinopropylamine (242 mg, 245 μL, 1.68 mmol) was added dropwise to the stirring mixture. The reaction tube was sealed and treated for 30 min at 125° C. in the microwave. The solution was then basified with potassium carbonate and extracted with chloroform (3×5 ml). The organic phases were collected, dried over sodium sulphate and evaporated. The residue obtained was purified through column chromatography using as eluent a mixture of CH₂Cl₂/MeOH 96/40. Yield 3a (107 mg, 0.18 mmol, 35%)¹H NMR (400 MHz, CDCl₃) δ 1.95-1.97 (m, 4H), 2.40-2.43 (m, 8H), 2.52-2.53 (m, 4H), 3.51-3.54 (m, 8H), 4.28-4.33 (q, 4H), 8.77 (d, 1H, J=8 Hz), 8.82 (d, 1H, J=7.6 Hz), 8.935 (s, 1H). ¹³C NMR (100 MHz, CDCl3, TMS) δ 29.3, 29.7, 31.2, 38.1, 53.5, 56.4, 56.5, 59.5, 66.9, 126.0, 126.1, 126.7, 126.9, 128.6, 130.6, 130.8, 131.6, 138.4, 161.1, 161.8, 161.9, 162.5. HRMS (ES⁺) calculated C₂₈H₃₁BrN₄O₆[M+H]⁺ 600.1543. found: 600.1536.

### 2,7-bis(3-morpholinopropyl)-4-((2-(pyrrolidin-1-yl)ethyl)amino)benzo[Imn][3,8]phenanthroline-1,3,6,8(2H,7H)-tetraone (4c)

Compound 3a (0.15 g, 0.25 mmol), 1-(2-aminoethyl)pirrolidine (0.06 mL, 0.51 mmol), and NMP (1 mL) were suspended in a microwave reaction vessel. The reaction tube was sealed and treated for 25 min at 130° C. in the microwave. After having been cooled to room temperature, the solvent was concentrated and the crude mixture was purified by column chromatography, using as eluent a mixture of CH₂Cl₂/MeOH/NH₃ 95/5/0.4. Compound 4c was obtained as a red oil (118 mg, 0.186 mmol, 75% yield). ¹H NMR (400 MHz, CDCl₃, TMS) δ 2.019-2.040 (m, 8H), 2.683-2.782 (m, 12H), 3.195 (t, 4H, J=6.4 Hz), 3.32 (t, 2H, J=6.6 Hz), 3.708 (t, 4H, J=4.8 Hz), 3.764 (t, 4H, J=4.6 Hz), 4.009-4.024 (m, 2H), 4.22 (t, 4H, J=7 Hz), 8.237 (s, 1H), 8.28 (d, 1H, J=7.6 Hz), 8.56 (d, 1H, J=8 Hz), 10.174 (t, 1H, exch D₂O, J=4.2 Hz). ¹³CNMR (100 MHz, CDCl₃) δ 23.3, 23.6, 23.7, 38.2, 38.9, 40.1, 52.6, 52.8, 53.9, 53.9, 55.8, 65.6, 65.8, 100.7, 119.1, 119.5, 123.5, 124.9, 126.1, 128.0, 129.2, 131.4, 151.8, 162.8, 163.2, 165.9, 166.5. HRMS (ES⁺) calculated for (M+H)⁺ C₃₄H₄₄N₆O₆ 633.3389, found 633.3401.

### Salt Formation

4c was then converted in a salt in order to enhance its aqueous solubility. Different salts were obtained, their solubility at 5 mg/ml and the pH of the resulting aqueous solution were evaluated. The results are reported in Table 2.

TABLE 2
Solubility data for different 4c salts.

| 4c salt type | Solubility 5 mg/ml | pH solution 5 mg/ml |
|---|---|---|
| Monohydrochloride | Low solubility | 7.4 |
| Dihydrochloride | Low solubility | 6.86 |
| Trihydrochloride | Soluble | 3.99 |
| Monoformate | Not soluble | |
| Diformate | Not soluble | |
| Triformate | Soluble | 5.64 |
| Monoacetate | Not soluble | |
| Diacetate | Not soluble | |
| Triacetate | Not soluble | |
| Monohydrochloride | Soluble | 6.95 |
| monoformate | | |

### Biophysical and Cell Biology Data

FRET Assay.

The ability of the naphthalene diimide compounds to stabilize quadruplex DNA sequences was investigated using a fluorescence resonance energy transfer (FRET) assay modified to be used as a high-throughput screen in a 96-well format. Several quadruplex sequences were studied (including the human telomeric G-quadruplex DNA sequence 5′-FAM-d(GGG[TTAGGG]₃)-TAMRA-3′ and the duplex sequence 5′-FAM-dTATAGCTATA-HEG-TATAGCTATA-TAMRA-3′ (HEG linker: [(—CH₂—CH₂—O—)₆.

The labelled oligonucleotides have attached to them the donor fluorophore FAM: 6-carboxyfluorescein and the acceptor fluorophore TAMRA: 6-carboxytetramethylrhodamine. The FRET probe sequences were diluted from stock to the correct concentration (400 nM) in a 60 mM potassium cacodylate buffer (pH 7.4) and then annealed by heating to 85° C. for 10 min, followed by cooling to room temperature in the heating block. The compound was stored as a 10 mM stock solution in DMSO; final solutions (at 2× concentration) were prepared using 10 mM HCl in the initial 1:10 dilution, after which 60 mM potassium cacodylate buffer (pH 7.4) was used in all subsequent steps. The maximum HCl concentration in the reaction volume (at a ligand concentration of 20 μM) is thus 200 μM, well within the range of the buffer used. Relevant controls were also performed to check for interference with the assay. 96-Well plates (MJ Research, Waltham, Mass.) were prepared by aliquoting 50 μl of the annealed DNA into each well, followed by 50 μl of the compound solutions. Measurements were made on a DNA Engine Opticon (MJ Research) with excitation at 450-495 nm and detection at 515-545 nm. Fluorescence readings were taken at intervals of 0.5° C. in the range 30-100° C., with a constant temperature being maintained for 30 s prior to each reading to ensure a stable value. Final analysis of the data was carried out using a script written in the program Origin 7.0 (OriginLab Corp., Northampton, Mass.). The advanced curve-fitting function in Origin 7.0 was used for calculation of ΔTₘ values. All determinations were performed in triplicate or better. Esds in ΔTₘ are ±0.1° C.

TABLE 3
ΔTₘ values (° C.) for FRET analyses of compounds
4a-j at 1 μM concentrations with a series of G-rich sequences:
human telomeric (F21T), two sequences from the HSP90 promoter
Hsp90A, Hsp90B (Heat Shock Protein 90), two sequences from the
promoter region of the kras gene, one from the promoter region of
the bcl-2 gene, and Tloop (representing duplex DNA). Esds are
from triplicate measurements and average 0.3° C.

| | F21T | Hsp90A | Hsp90B | Kras21 | Kras32 | Bcl-2 | Tloop |
|---|---|---|---|---|---|---|---|
| 4a | 14.3 | 19.8 | 16.5 | 9.8 | 6.1 | 15.8 | 0.8 |
| 4b | 12.3 | 18.1 | 15.0 | 11.0 | 6.1 | 15.1 | 0.4 |
| 4c | 11.8 | 15.7 | 12.7 | 11.0 | 9.6 | 13.3 | 0.6 |
| 4d | <2 | 2.5 | <2 | 3.0 | 1.5 | 2.8 | 0 |
| 4e | 15.9 | 20.6 | 16.9 | 11.7 | 6.6 | 16.1 | 0.2 |
| 4f | 9.8 | 9.1 | 8.7 | 4.8 | 2.2 | 5.9 | 0.6 |
| 4g | 6.0 | 11.0 | 9.4 | 2.4 | 1.5 | 5.8 | 1.5 |
| 4h | 1.4 | 3.9 | 6.8 | 3.4 | 1.2 | 4.2 | 0 |
| 4i | <2 | 2.7 | 4.5 | 0.7 | 0.2 | 2.4 | 0 |
| 4j | 9 | 15.7 | 13.8 | 6.1 | 3.0 | 10.0 | 0.1 |
| MM41 | 26.6 | 33.1 | 28.6 | 22.5 | 19.8 | 26.4 | 4.9 |

Cell Culture and Cytotoxicity Testing.

Human cancer cell lines and the somatic human cell line WI-38 (lung fibroblast) were purchased from the American Type Culture Collection. Cell lines were maintained in appropriate medium supplemented with 10% foetal bovine serum (Invitrogen, UK), 2 mM L-glutamine (Invitrogen, Netherlands), and other components as specified by the suppliers. Cell lines were maintained at 37° C., 5% CO₂ and routinely passaged. Drugs were dissolved in DMSO and filtered through 0.22 μm pore-size filter units before addition to cell line appropriate media. Cellular growth inhibition was measured using the sulforhodamine B (SRB) assay in 96 well plates. Fifty percent inhibitory concentrations (IC₅₀) were determined by taking the mean absorbance at 540 nm for each drug concentration expressed as a percentage of the absorbance of untreated control wells.

For qRT-PCR analysis, Mia-PaCa2 cells were seeded to a density equivalent to that used for IC₅₀ determinations in T75 culture flasks and grown for 24 h in 10 ml DMEM to allow attachment before addition of compound 3d to the culture medium. Following compound exposure, cells were washed twice in PBS, harvested by trypsinisation, collected by centrifugation (300 g, 5 min, 4° C.) and re-suspended in RLT buffer (Qiagen). Samples were homogenised using QIAshredder spin columns and stored at −80° C. prior to RNA extraction. Three biological replicates were performed on separate days.

TABLE 4
short-term 96 hr IC₅₀ values (in μM) for compounds
4a-j and MM41 in a cancer cell line panel, comprising MCF7
(breast), A549 (lung cancer), Mia-PaCa2/Panc1 (pancreatic
cancer), ALT (Alternative Lengthening of Telomeres) and WI38
(lung fibroblast) cell lines. Esds average 0.25 μM. (*) indicates
concentration of 5 μM instead of 1 μM. MM41 is the
di-morpholino-di-N-methyl-piperazine naphthalene diimide
derivative highlighted in Micco et al J Med Chem, 2013.

| | A549 | MCF7 | MiaPaCa2 | Panc1 | ALT | WI38 |
|---|---|---|---|---|---|---|
| 4a | 0.067 | 0.357 | 0.059 | 0.045 | 0.224 | 1.83 |
| 4b | 0.086 | 0.316 | 0.048 | 0.046 | 0.085 | 1.49 |
| 4c | 0.024 | 0.159 | 0.012 | 0.022 | 0.093 | 1.19 |
| 4d | 0.130 | 1.070 | 0.220 | 0.340 | 1.29 | 2.24 |
| 4e | 0.026 | 0.222 | 0.036 | 0.033 | 0.089 | 1.22 |
| 4f | 0.198 | 1.110 | 0.108 | 0.084 | 0.535 | 5.33 |
| 4g | 2.18 | >25 | 0.206 | 0.220 | 10.874 | 17.65 |
| 4h | 0.146 | 1.03 | 0.139 | 0.808 | 0.71 | 1.88 |
| 4i | 0.825 | 3.33 | 1.085 | 0.909 | 2.67 | 5.53 |
| 4j | 0.092 | 1.538 | 0.059 | 0.163 | 0.451 | 1.65 |
| MM41 | 0.019 | 0.070 | 0.011 | 0.003 | 0.063 | 0.230 |

### Antitumour Activity

### Protocol

Study the therapeutic effect of compound 4c (MM41-tri) in comparison to MM41 and Gemcitabine in CD1 nude mice carrying a subcutaneous xenograft of the pancreatic tumour cell line MiaPaCa-2 (5×10⁶ cells in the right flank) grown with Matrigel.

### Experimental Outline

1 Inoculate SC 5×10⁶ Miapaca-2 cells in the right flank (unsupplemented RPMI+Matrigel).
2 Wait for tumours to establish subcutaneously (approx. 11 days).
3 Measure size of the tumours weekly until they reach a mean size of 0.1 cm³. At this stage of the development of the tumours, mice are ready to begin the therapy study.
4 Re group mice to form 5 therapeutic groups constituted of 8 mice each, carrying approximately 0.1 cm³ average of the tumour size per group.
5 Administered corresponding dose of MM41 or compound 4c or Gemcitabine by IV.
   Group 1: 8 mice treated with 15 mg/Kg of Gemcitabine twice weekly dose.
   Group 2: 8 mice treated with 15 mg/Kg of MM41 (tetrasubstituted ND compound) twice weekly dose.
   Group 3: 8 mice treated with 15 mg/Kg of Compound 4c twice weekly dose.
   Group 4: 8 mice treated with 10 mg/Kg of Compound 4c twice weekly dose.
   Group 5: 8 control mice treated with saline only twice weekly.

The tumour growth curve is shown in FIG. 1. Compound 4c (marked as MM41-tri) at 15 mg/kg is showing a highly significant anti-tumour response.

MM41 is the di-morpholino-di-N-methyl-piperazine naphthalene diimide derivative highlighted in Micco et al J Med Chem). 2013. Animals did not have any significant weight loss on treatment.

### Gene Expression

### Methods

### Cell Culture

Cell lines were purchased from American Type Culture Collection (ATCC) and stored under liquid nitrogen. Cells were maintained in monolayer culture T75 (75 cm2 flasks) in humidified 5% CO2 atmosphere at 37° C. Cells were maintained in appropriate medium according to (ATCC) with necessary supplements including L-glutamine (Thermo Fisher Scientific Cat. No. 25030024), penicillin-streptomycin solution Hybrid-Max (Sigma Cat. No. P7539) and fetal bovine serum (FBS, Thermo Fisher Scientific Cat. No. 10270106). Cells were passaged at 80-90% confluence.

### RNA Extraction

MIA-PaCa2 cells were seeded 48 hours prior to incubation, in MatTek 35 mm glass bottom dishes. Three incubation times for the compound 4c were examined: 0, 6 and 24 hrs. For RNA extraction cells (a maximum of 1×107 cells) were harvested by trypsinization following rinsing the cells twice in 1×PBS (without calcium chloride and magnesium chloride, purchased from Sigma Cat. No. D1408). After neutralising the medium with an equal volume of medium to trypsin cells were transferred to a sterile centrifuge tube for centrifugation for 4 minutes at 400 rpm at room temperate in order to pellet the cells. After removing the medium and trypsin following centrifugation RNA was extracted from the pellet of cells using the RNeasy Mini Kit by QIAGEN (Cat. no. 74104). RNA quality was checked by use of the NanoDrop 2000 Spectrophotometer, and a minimum quantity of ca 500 ng of RNA at >25 ng/μl concentration per replicate was verified.

RNA-Seq sequencing studies were performed by the UCL Genomics Facility using an Illumina NextSeq 500 instrument, and data obtained on RNA expression levels of all individual genes in the genomes of the cells analysed, relative to the control cells at 0 hr exposure. Four replicates were used for each time-point, thus a total of 12 individual genomes were analysed and statistics obtained for each time-point.

Gene Expression Analyses—Changes in Gene Expression Following Treatment with Compound 4c

Data has been taken from RNA-seq data from treated MIA-PaCa2 cells, showing log-fold expression changes for genes selected from those showing the most significant down-regulation of expression as measured by RNA levels.

TABLE 5
Selection from the most down-regulated genes (log-fold changes
relative to t = 0 hrs, ie untreated cells, are shown)

| | | | No. of |
|---|---|---|---|
| | 4c at 6 hrs | 4c at 24 hrs | promoter G4s |
| PRDM16 | −2.8 | −3.5 | 5 |
| CBFA2T3 | −2.8 | −2.8 | 7 |
| TREX1 | −8.3 | −2.5 | 2 |
| SHANK2 | −1.9 | −2.0 | 2 |
| TP73 | −2.4 | −1.2 | 5 |
| ZNF469 | −3.4 | −3.2 | 1 |

TABLE 6
Function of the selected most down-regulated genes

| | | Implicated in human |
|---|---|---|
| Gene | Function | pancreatic cancer? |
| PRDM16 | Transcription regulator of TGFB, | Y |
| | SMAD pathways | |
| CBFA2T3 | Transcription regulator of HDAC | Y |
| | pathways | |
| TREX1 | Exonuclease in ATM/ATR/RAD | Y |
| | repair pathways | |
| SHANK2 | Axon guidance and SRC pathways | Y |
| TP73 | Cell cycle and DNA repair regulation | Y |
| ZNF469 | Transcription factor and pre-mRNA | Y |

TABLE 7
Selection of some of the most up-regulated genes (log-fold changes
relative to t = 0 hrs, ie untreated cells, are shown)

| | 4c at | 4c at | | No. of |
|---|---|---|---|---|
| Gene | 6 hrs | 24 hrs | Function | promoter G4s |
| ITGAM | 7.0 | 2.6 | Integrin alpha M chain | 0 |
| ITGA1 | 4.5 | 0.4 | Integrin subunit | 0 |
| ST14 | 5.6 | 2.5 | Suppressor of tumorigenicity | 0 |
| FLG | 4.0 | 0.7 | Filament-associated protein | 0 |
| AVIL | 3.0 | 0.5 | Actin regulatory protein | 0 |

The analysis of changes in gene expression shows a consistent pattern of changes at the two time-points, with the down-regulated genes (Table 5 above) being generally coding for proteins involved in transcriptional activation, GTPase activity and cancer pathways, including DNA damage responses. It is striking that the most strongly down-regulated genes tend to have putative DNA quadruplex sequences within their promoter regions. By contrast the up-regulated genes (Table 7 above) tend to code for proteins involved in membrane and extracellular matrix structure and function, and have significant under-representation of putative quadruplex sequences in their promoters. The number of putative quadruplex sequences in each gene promoter has been estimated from the primary sequence using the Ensembl genome browser (version 86, http://www.ensembl.org/index.html).

Several of the genes most down-regulated in the analysis above (for example, PRDM16, CBFA2T3 and SHANK2) have been previously reported as being associated with improved patient survival in pancreatic cancer when found to be methylated (Thompson M J, Rubbi L, Dawson D W, Donahue T R, Pellegrini M (2015) Pancreatic Cancer Patient Survival Correlates with DNA Methylation of Pancreas Development Genes. PLoS ONE 10(6): e0128814). The gene products of the TREX1 and TP73 genes are involved in DNA repair: it has been reported that polymorphisms in these genes also correlate with patient survival in pancreatic cancer (Dong X, Li Y, Hess K R, Abbruzzese J L, Li D. (2011) DNA Mismatch Repair Gene Polymorphisms Affect Survival in Pancreatic Cancer. Oncologist 16(1), 61-70).

Previous bioinformatics studies have suggested that the human genome contains between 300,000 and 700,000 potential quadruplex-forming sequences (Todd A K, Johnston M, Neidle S. (2005) Highly prevalent putative quadruplex sequence motifs in human DNA. Nucleic Acids Research 33(9), 2901-2907; Huppert J L, Balasubramanian S. (2005) Prevalence of quadruplexes in the human genome. Nucleic Acids Research 33(9), 2908-2916; Kwok C K, Marsico G, Sahakyan A B, Chambers V S, Balasubramanian S. (2016) rG4-seq reveals widespread formation of G-quadruplex structures in the human transcriptome. Nature Methods 13(10), 841-844). However very recent studies of quadruplex formation in active chromatin in cells (Hansel-Hertsch R, Beraldi D, Lensing S V, Marsico G, Zyner K, Parry A, Di Antonio M, Pike J, Kimura H, Narita M, Tannahill D, Balasubramanian S. (2016) G-quadruplex structures mark human regulatory chromatin. Nature Genetics 48(10), 1267-1272), has shown that only about 10,000 quadruplexes exist in human chromatin, and are enriched in the promoter regions, primarily of genes involved in human cancer. This and findings of the presence of only a small number of folded RNA quadruplexes in eukaryotic cells (Guo J U, Bartel D P. (2016) RNA G-quadruplexes are globally unfolded in eukaryotic cells and depleted in bacteria. Science 353(6306). pii: aaf5371), support the hypothesis that quadruplex-selective targeting is feasible in the treatment of human cancers.

Surprisingly it has been found that MIA-PaCa2 pancreatic cancer cells when treated with compound 4c, show changes in gene expression in a comparatively small number of genes, the majority of which have putative quadruplex-forming sequences in their promoters, consistent with the concept that the compound is selectively targeting these nuclear genes.

Surprisingly, a number of these targeted genes are associated with increased patient survival where their expression in humans is down-regulated, supporting the concept that compounds in this disclosure such as compound 4c, may have utility in the treatment of human cancers.

1. A compound comprising the formula I 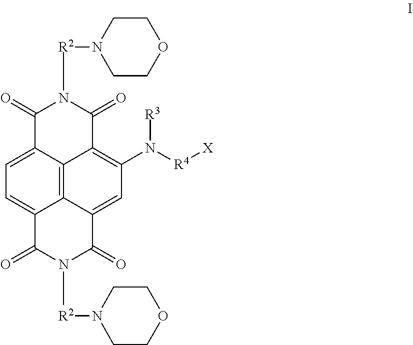
R² are the same and are selected from the group consisting of straight and branched, C₁₋₆-alkyanediyl;
R³ is selected from the group consisting of H and C₁₋₆ alkyl;
R⁴ is selected from the group consisting of straight and branched C₁₋₆-alkanediyl and C₇₋₁₂ aralkane-diyl;
X is selected from the group consisting of halo, OR¹, NR⁵₂, CONR⁶₂, COOR⁷, H and COR⁸,
R¹ is selected from the group consisting of H, optionally substituted C₁₋₆ alkyl, optionally substituted C₅₋₇ cycloalkyl, C₅₋₇ heterocycloalkyl and aryl;
each R⁵ is independently selected from the group consisting of H, C₁₋₆ alkyl, aryl and, C₇₋₁₂-arylkyl, or the groups R⁵ together with the N-atom to which they are attached form a N-containing, saturated heterocyclic group;
each R⁶ is independently selected from H and C₁₋₆ alkyl groups, or the groups R⁶ together with the N atom to which they are attached from a 5-17 membered heterocyclic ring;
R⁷ is selected from the group consisting of optionally substituted C₁₋₆ alkyl, C₇₋₁₂ aralkyl, and aryl;
R⁸ is selected from the group consisting of optionally substituted C₁₋₆-alkyl, C₇₋₁₂-aralkyl and aryl;
provided that when X is 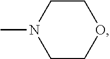
R⁴ is different than R²;
and salts, hydrates and solvates thereof.

2. The compound of claim 1, wherein R² is straight chain C₂₋₄ alkanediyl.

3. The compound of claim 1, wherein R³ is H.

4. The compound of claim 1, wherein R⁴ is a straight-chain C₂₋₄-alkanediyl.

5. The compound of claim 1, wherein X is NR⁵₂.

6. The compound of claim 5, wherein the R⁵ groups together with the nitrogen atom to which they are attached form a heterocyclic group selected from 4-methyl piperazine-1-yl, morpholine-4-yl, pyrrolidin-1-yl, pyridin-2-yl and piperidin-1-yl.

7. A composition comprising a compound of claim 1 in combination with a diluent.

8. (canceled)

9. The composition according to claim 7, further comprising a compound of the formula II 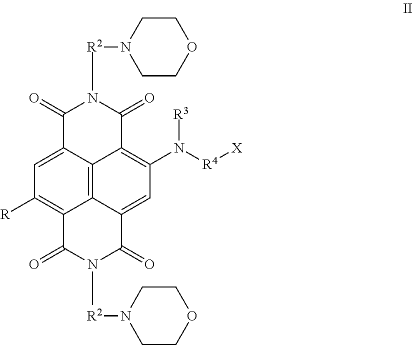
wherein R is NH₂ or N(R¹³)R¹⁴X¹, wherein
R¹³ is selected from the same groups as R³,
R¹⁴ is selected from the same groups as R⁴,
X¹ is selected from the same groups as X,
provided that the compound of formula II is present in an amount no higher than 50% by weight of the compound of formula I.

10. A method of treating a solid tumour in an animal in need thereof comprising administering a therapeutically effective amount of a compound of claim 1.

11. The method of claim 10, wherein the solid tumour is a pancreatic tumour.

12. A method of synthesis of a substituted naphthalene diimide compound comprising the step of reacting a compound of formula III 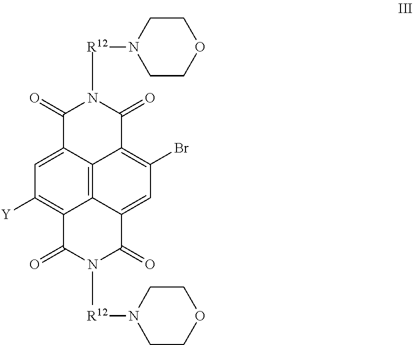
wherein Br is bromo;
Y is H or Br;
R¹² are the same and are selected from the group consisting of straight and branched C₁₋₆ alkenediyl;
in a nucleophilic substitution reaction with an amine reagent of formula IV
R¹³HNR¹⁴X² IV
wherein R¹³ is selected from the group consisting of H and C₁₋₆ alkyl;
R¹⁴ is selected from the group consisting of straight and branched C₁₋₆ alkanediyl and C₇₋₁₂ aralkanediyl;
X² is selected from the group consisting of halo, R¹¹, NR¹⁵₂, CONR¹⁶₂, COOR¹⁷, SH and COR¹⁸;
R¹¹ is selected from the group consisting of H, optionally substituted C₁₋₆ alkyl, optionally substituted C₅₋₇ cycloalkyl, C₅₋₇ heterocycloalkyl and aryl;
each R¹⁵ is independently selected from the group consisting of H, C₁₋₆ alkyl, aryl and C₇₋₁₂ aralkyl, or the groups R¹⁵ together with the N-atom to which they are attached form a saturated heterocyclic ring of 5-7 atoms;
each R¹⁶ is independently selected from the group consisting of H and C₁₋₆ alkyl groups, or the groups R¹⁶ together with the N atom to which they are attached form a 5-7 membered heterocyclic ring;
R¹⁷ is selected from the group consisting of optionally substituted C₁₋₆ alkyl, C₇₋₁₂ aralkyl and aryl;
R¹⁸ is selected from the group consisting of optionally substituted C₁₋₆ alkyl, C₇₋₁₂ aralkyl and aryl;
whereby the Br atom, or when Y is Br one of the Br atoms or each Br atom, is substituted by the nucleophilic amine nitrogen of the amine reagent to form the substituted naphthalene diimide compound.

13. The method of claim 12, wherein Y is Br.

14. The method of claim 13, wherein one of the Br atoms is substituted by the amine nitrogen and the other is not reacted or is substituted by H.

15. The method of claim 12, wherein Y is H.

16. The method of claim 12, wherein R¹³ is H.

17. The method of claim 12, wherein R¹⁴ is selected from the group consisting of C₂₋₄ alkanediyl.

18. The method of claim 12, wherein X² is NR¹⁵₂.

19. The method of claim 18, wherein the groups R¹⁵ together with the N-atom to which they are attached form a heterocyclic ring selected from N-methyl piperazine, pyrrolidone, 4-morpholino and piperidine.

20-24. (canceled)

25. The method of claim 10, wherein the growth of the solid tumour is inhibited, or the size of the solid tumour is reduced.
